(19) United States
(12) Patent Application Publication
(10) Pub. No.: US 2005/0202545 A1
(43) Pub. Date: Sep. 15, 2005

(54) Methods for producing optically active alpha-hydroxy amides

(76) Inventors: Kohji Ishihara, Okayama (JP); Hiroaki Yamamoto, Ibaraki (JP)

Correspondence Address: LAHIVE & COCKFIELD, LLP., 28 STATE STREET, BOSTON, MA, 02109, US

(21) Appl. No.: 11/048,459

(22) Filed: Jan. 31, 2005

(30) Foreign Application Priority Data
Feb. 5, 2004 (JP) 2004-029113

(51) Int. Cl. C12P 13/00; C12N 9/06

(52) U.S. Cl. 435/128; 435/191

(57) ABSTRACT

An objective of the present invention is to provide efficient methods for producing (R)-2-chloromandelamide with high optical purity. Another objective of the present invention is to provide novel methods for producing α-ketoamide reductases that reduce 2-chlorobenzoyl formamide to (R)-2-chloromandelamide with high optical purity, using NADPH as the coenzyme.

An enzyme exhibiting high stereoselectivity was purified from a number of Saccharomyces cerevisiae enzymes with 2-chlorobenzoyl formamide-reducing activity, and the biochemical properties of the purified enzyme were analyzed. The analysis of a partial internal amino acid sequence of the purified enzyme revealed that the enzyme may be encoded by the putative open reading frame (ORF) YDL124w reported in the genome analysis. YDL124w was cloned and expressed in E. coli, and was subsequently shown to encode the α-ketoamide reductase. It was found that these resulting transformants facilitated the production of (R)-2-chloromandelamide from 2-chlorobenzoyl formamide.

### FIELD OF THE INVENTION

The present invention relates to methods for producing optically active alcohols. More specifically, the present invention relates to methods for producing optically active α-hydroxy amides, particularly (R)-2-chloromandelamide, using reduced β-nicotinamide adenine dinucleotide phosphate (hereinafter abbreviated as NADPH)-dependent α-ketoamide reductases.

### BACKGROUND OF THE INVENTION

Conventional methods for producing (R)-mandelamide without a halogen substituent in the benzene ring include (1) reacting (R)-mandelic acid with thionyl chloride to convert it to an acid chloride, and then reacting the acid chloride with ammonia (Phytochemistry 34, 433-436 (1993)); (2) converting (R)-mandelic acid to a methyl ester or the like, and then reacting it with ammonia (Agri. Biol. Chem. 53, 165-174 (1989)); (3) column resolution of racemic mandelamide (Chem. Ber. 116, 3611-3617 (1983)); and (4) asymmetrically reducing benzoyl formamide using baker's yeast (Aust. J. Chem. 29, 2459-2467 (1976)). However, even if the starting material (R)-mandelic acid can be converted into (R)-mandelic acid with a halogen substituent in the benzene ring, the first two of these methods are not suitable for industrial production, due to the expense of the starting material coupled with the potential for optical purity to be decreased during the reaction. Production of (R) -mandelamide by column resolution might be used to isolate a (R) -mandelamide derivative with a halogen substituent in the benzene ring, from a racemic mandelamide derivative with a halogen substituent in the benzene ring, but the maximal yield is no more than 50% because of the unnecessary S-isomer, and thus this technique is disadvantageous as an industrial method.

### SUMMARY OF THE INVENTION

An objective of the present invention is to provide efficient methods for producing compounds of high optical purity, which can be used to produce optically active alcohols, more specifically optically active α-hydroxy amides, particularly (R)-2-chloromandelamide, using NADPH-dependent α-ketoamide reductases.

To develop more efficient methods for producing (R)-2-chloromandelamide, the present inventors focused on the method of converting benzoyl formamide to (R) -mandelamide via asymmetrical reduction, which theoretically allows 100% of the starting material to be converted to the desired compound. The present inventors performed intensive studies to establish methods for producing (R) -mandelamide derivatives with a halogen substituent in the benzene ring, particularly (R)-2-chloromandelamide, which can be used in pharmaceutical products. They found and reported that (R)-2-chloromandelamide can be produced more efficiently by asymmetrically reducing 2-chlorobenzoyl formamide using baker's yeast and tropinone reductase-I (Japanese Patent Application No. 2002-234688).

In the present invention, baker's yeast cells (Oriental Yeast Co., Ltd.) were lysed, and an enzyme responsible for the asymmetric reduction of 2-chlorobenzoyl formamide was purified from the resulting cell-free extract, until the purified product gave a single band by electrophoresis. Then, the various properties of the enzyme were analyzed. The optical purity of (R)-2-chloromandelamide produced by the reduction varied among fractions obtained during the chromatography purification process. This suggests that baker's yeast contains a number of α-ketoamide reductases. with differing stereoselectivities. A purified enzyme was obtained by recovering and purifying only the active fractions that have high optical purity, and this enzyme was used to produce (R)-2-chloromandelamide of 99% or higher purity. By comparison, the baker's yeast described in Japanese Patent Application No. 2002-234688, yielded (R)-2-chloromandelamide by asymmetric reduction with an optical purity of 95.3%.

In addition to the asymmetric reductions of various α-ketoamides to selective R-isomers, this purified enzyme surprisingly catalysed various α-ketoesters to produce (R)-α-hydroxyesters of exceedingly high optical purity.

The molecular weight (approximately 33,000 by gel filtration; approximately 36,000 by sodium dodecyl sulfate-gel electrophoresis (SDS-PAGE)) and the substrate specificity ((R)-α-hydroxyesters are produced -by incubating various α-ketoesters with the enzyme) of the enzyme suggest that it is identical to the protein YKER-IV, the α-ketoester reductase of baker's yeast reported by Nakamura et al. (the molecular weight of this enzyme was 31,000 by gel filtration, and 39,000 by SDS-PAGE; Kaoru Nakamura, Shin-ichi Kondo, Yasushi Kawai, Nobuyoshi Nakajima, and Atsuyoshi Ohno, Biosci. Biotech. Biochem. 58, 2236-2240 (1994)). Nakamura et al. have reported that baker's yeast contains at least seven α-ketoester-reducing enzymes: YKER-I, -II, -III, -IV, -V, -VI, and -VII. It has been unclear which, if any, of these seven α-ketoester reductases are able to reduce α-ketoamides and what stereoselectivity the enzyme exhibits. In this context, it is quite amazing that the enzyme of the present invention has both α-ketoamide reducing activity and α-ketoester reducing activity. The amino acid sequence of YKER-IV has not yet been reported, possibly because the N-terminal amino acid is blocked. Thus, it has so far been impossible to clearly identify that YKER-IV is identical to the enzyme of the present invention.

The N-terminal amino acid sequence of the purified enzyme was analyzed to clone the gene encoding the enzyme. However, the inventors failed to determine the amino acid sequence, possibly because the N-terminus is blocked.

Then, the present inventors subjected the purified enzyme to SDS-PAGE, excised a gel piece containing the enzyme, and trypsinized the enzyme in the gel. The resulting fragments of the enzyme were fractionated by reversed-phase chromatography. The inventors succeeded in determining the internal sequence of the purified enzyme by analyzing the amino acid sequences of the fractionated peptide fragments.

The amino acid sequence determined was analyzed by a homology search against the disclosed genomic sequence of Saccharomyces cerevisiae and others. The sequence agreed completely with a portion of the amino acid sequence deduced from the putative open reading frame (ORF) reported as YDL124w, whose function has remained unknown.

Polymerase Chain Reaction (PCR) primers were designed based on the nucleotide sequence of YDL124w, to amplify only the ORF of the gene. PCR was carried out using these primers and the chromosomal DNA of Saccharomyces cerevisiae as a template, to verify that YDL124w was the gene encoding the α-ketoamide reductase. The resulting DNA fragment was inserted into the E. coli expression vector, pSE420D (Unexamined Published Japanese Patent Application No. (JP-A) 2000-189170), to construct the expression plasmid pSE-YDL1. E. coli cells were transformed with this plasmid, and the resulting transformants were cultured. Cell-free extracts were prepared and assessed for enzymatic activity. The extracts showed exceedingly high α-ketoamide reductase activity.

In a separate experiment, cell-free extracts prepared from E. coli cells transformed with the plasmid pSE-YDL1, were centrifuged after addition of ammonium sulfate. Precipitated fractions of 70% ammonium sulfate saturation obtained were dialyzed to prepare crude enzyme solutions. The solutions were then used to perform the reduction reaction. As with the purified enzyme, the crude enzyme solution resulted in the production of (R)-2-chloromandelamide with 99% ee or higher purity quantitatively from 2-chlorobenzoyl formamide, and ethyl (R)-2-hydroxyisovalerate with 99% ee or higher purity was produced quantitatively from 2-oxo isovalerate ethyl ester. These results indicate that YDL124w encodes the α-ketoamide reductase.

There are currently no reports describing the functions of the protein encoded by YDL124w. Thus, it has not been predicted that the protein asymmetrically reduces α-ketoamides to (R)-α-hydroxy amides with exceedingly high optical purity.

Specifically, the present invention relates to methods for producing optically active alcohols, and provides:
[1] An α-ketoamide reductase comprising the physicochemical properties of:
   (1) activity:
reduces a ketone using NADPH as coenzyme to produce an optically active alcohol;
   2substrate specificity:
      (a) uses NADPH as coenzyme in reduction reactions; and
      (b) reduces 2-chlorobenzoyl formamide to produce (R)-2-chloromandelamide with an optical purity of 98% ee or higher;
   (3) molecular weight:
approximately 33,000 by gel filtration, and approximately 36,000 by SDS-PAGE;
   (4) optimal pH:
pH 5.5 to 6.5; and
   (5) optimal temperature:
35 to 47° C.
[2] A method for producing an optically active alcohol, wherein the method comprises reacting a ketone with the α-ketoamide reductase according to [1], and wherein the produced optically active alcohol corresponds to the ketone.
[3] The method for producing an optically active alcohol according to [2], wherein the ketone is an α-ketoamide and the corresponding optically active alcohol is an optically active α-hydroxy amide.
[4] The method for producing an optically active alcohol according to [3], wherein the α-ketoamide is a benzoyl formamide derivative and the corresponding optically active α-hydroxy amide is an (R)-mandelamide derivative.
[5] A method for producing an optically active alcohol, wherein the method comprises reacting a ketone with a transformant expressing an α-ketoamide reductase encoded by a polynucleotide according to any one of (a) to (e), or a processed product thereof,
   (a) a polynucleotide comprising the nucleotide sequence of SEQ ID NO: 1;
   (b) a polynucleotide encoding a protein comprising the amino acid sequence of SEQ ID NO: 2;
   (c) a polynucleotide encoding a protein comprising the amino acid sequence of SEQ ID NO: 2, wherein one or more amino acids have been substituted, deleted, inserted, and/or added;
   (d) a polynucleotide that hybridizes under stringent conditions to a DNA comprising the nucleotide sequence of SEQ ID NO: 1; and
   (e) a polynucleotide encoding an amino acid sequence exhibiting a homology of 50% or higher to the amino acid sequence of SEQ ID NO: 2, and wherein the produced optically active alcohol corresponds to the ketone.
[6] The method for producing an optically active alcohol according to [5], wherein the ketone is an α-ketoamide or α-ketoester and the corresponding optically active alcohol is an (R)-α-hydroxy amide or (R)-α-hydroxyester.
[7] The method for producing an optically active alcohol according to [6], wherein the α-ketoamide is a benzoyl formamide derivative and the (R)-α-hydroxy amide is an (R)-mandelamide derivative.
[8] A method for producing an α-ketoamide reductase, which comprises. the step of culturing a transformant expressing the α-ketoamide reductase according to [1], or an α-ketoamide reductase encoded by any one of:
   (a) a polynucleotide comprising the nucleotide sequence of SEQ ID NO: 1;
   (b) a polynucleotide encoding a protein comprising the amino acid sequence of SEQ ID NO: 2;
   (c) a polynucleotide encoding a protein comprising the amino acid sequence of SEQ ID NO: 2, wherein one or more amino acids have been substituted, deleted, inserted, and/or added;
   (d) a polynucleotide that hybridizes under stringent conditions to a DNA comprising the nucleotide sequence of SEQ ID NO: 1; and
   (e) a polynucleotide encoding an amino acid sequence exhibiting 50% or higher homology to the amino acid sequence of SEQ ID NO: 2.

### BRIEF DESCRIPTION OF THE DRAWINGS

FIG. 1 is a photograph showing a pattern of SDS-PAGE. Lane A, molecular weight marker; lane B, the enzyme obtained in Example 1.

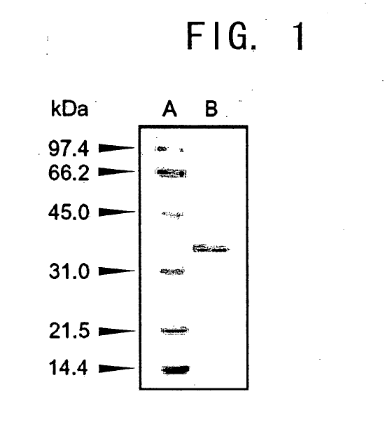

FIG. 2 is a diagram showing pH dependency of the 2-chlorobenzoyl formamide-reducing activity of the enzyme obtained in Example 1. Open square indicates sodium acetate buffer; open diamond, potassium phosphate buffer; open circle, Tris-hydrochloride buffer; and open triangle, glycine-potassium hydroxide buffer.

FIG. 3 is a diagram showing temperature dependency of the 2-chlorobenzoyl formamide-reducing activity of the enzyme obtained in Example 1.

FIG. 4 is a histogram showing pH stability of the enzyme obtained in Example 1.

FIG. 5 is a diagram showing thermostability of the enzyme obtained in Example 1.

FIG. 6 is a schematic illustration showing the construction of the plasmid pSE-YDL1, containing the Saccharomyces cerevisiae derived YDL124w gene as an insert. In the plasmid map, P(trc) refers to trc promoter; T(rrnB), rrnBT1T2 terminator; amp, beta-lactamase gene responsible for ampicillin resistance; ori, the replication origin; rop, ROP-protein gene; and laqIq, lactose repressor.

FIG. 7 is a schematic illustration showing the construction of the plasmid pSG-YDL1, containing the Saccharomyces cerevisiae derived YDL124w gene and the glucose dehydrogenase gene derived from Bacillus subtilis as inserts. In the plasmid map, P(trc) refers to trc promoter; T(rrnB), rrnBT1T2 terminator; amp, beta-lactamase gene responsible for ampicillin resistance; ori, the replication origin; rop, ROP-protein gene; laqIq, lactose repressor; and BsGlcDH, glucose dehydrogenase gene derived from Bacillus subtilis.

### DETAILED DESCRIPTION OF THE INVENTION

The α-ketoamide reductases to be used in the present invention have the properties described below in (1) to (5).
(1) Activity:
reduces a ketone using NADPH as coenzyme to produce an optically active alcohol
(2) Substrate specificity:
   (a) uses NADPH as coenzyme in reduction reactions; and
   (b) reduces 2-chlorobenzoyl formamide to produce (R)-2-chloromandelamide with an optical purity of 98% ee or higher
(3) Molecular weight:
approximately 33,000 by gel filtration using HiLoad 16/60 Superdex 200 (Amersham Biosciences), and approximately 36,000 by SDS-PAGE (12.5%)
(4) Optimal pH:
pH 5.5 to 6.5
(5) Optimal temperature:
35 to 47° C.

It is common knowledge in the art that the molecular weight of proteins determined by gel filtration or SDS-PAGE may vary depending on measurement conditions. Therefore, the word “approximately” in the phrases “approximately 33,000” and “approximately 36,000” means that the molecular weight of the protein falls within a range covering such variations. For example, a protein measured as approximately 33,000 may fall within a range of 30,000 to 36,000, and a protein measured as approximately 36,000 may fall within a range of 32,000 to 40,000, although it is not limited to these ranges. The potential molecular weight range of a protein measured as approximately 33,000 or approximately 36,000 should be determined according to common knowledge in the art.

In the present invention, α-ketoamide reductase activity can be tested, for example, by the method described below.

Method for determining 2-chlorobenzoyl formamide-reducing activity:

The enzyme is incubated in a reaction solution containing 100 mM potassium phosphate buffer (pH 6.5), 0.2 mM NADPH, and 1 mM 2-chlorobenzoyl formamide at 30° C. A decrease in NADPH concentration is assessed by the decrease in absorbance at 340 nm. 1 U is defined as the amount of enzyme required to catalyze the decrease of 1 μmol NADPH in one minute.

The α-ketoamide reductase described above can be purified from baker's yeast and other yeasts belonging to the genus Saccharomyces (particularly Saccharomyces cerevisiae) . Specifically, such yeasts include baker's yeast from Oriental Yeast Co., Ltd., Saccharomyces cerevisiae ATCC 208277 and ATCC 204505, and BJ2168.

The microorganisms described above may be cultured with. conventional media for yeast culture, such as YM medium (10 g/L glucose, 5 g/L peptone, 3 g/L yeast extract, and 3 g/L malt extract (pH 6.0)). The fungal cells are harvested after sufficient growth. The cells are lysed in a buffer containing a reducing agent, such as 2-mercaptoethanol, and a protease inhibitor, such as phenylmethanesulfonyl fluoride, to prepare the cell-free extract. The enzyme can be purified from the cell-free extract by using different methods in combination, as appropriate, including fractionation based on the differences of protein solubilities (precipitation with an organic solvent, salting out with ammonium sulfate, etc.), cation exchange chromatography, anion exchange chromatography, gel filtration, hydrophobic chromatography, affinity chromatography, including methods that utilize chelate, dye, antibody, or such. The enzyme can be purified until it gives a single band in electrophoresis, for example, by precipitation with 70% ammonium sulfate, DEAE-TOYOPEARL anion exchange chromatography, MonoQ anion exchange chromatography, phenyl-Superose hydrophobic chromatography, AF-Red TOYOPEARL affinity chromatography, and HiLoad 16/60 Superdex 200 gel filtration chromatography. Specifically, the enzyme can be purified by the method described in Example 1. The present invention also provides the α-ketoamide reductases that can be obtained by such methods. The specific activity of the α-ketoamide reductase provided by the above method is about 105 U/mg, and the enzyme contains the partial peptide comprising the amino acid sequence of SEQ ID NO: 3.

The present invention also provides α-ketoamide reductase comprising the amino acid sequence of SEQ ID NO: 2. The α-ketoamide reductases of the present invention comprise ,homologues of the protein containing the amino acid sequence of SEQ ID NO: 2.

The phrase “α-ketoamide reductase homologues of the present invention” refers to proteins that are functionally equivalent to the protein comprising the amino acid sequence of SEQ ID NO: 2 and comprise the amino acid sequence of SEQ ID NO: 2, in which one or more amino acids have been deleted, substituted, inserted, and/or added. For example, the amino acid sequence of SEQ ID NO: 2 has 100 or less , typically 50 or less, preferably 30 or less, more preferably 15 or less, still more preferably 10 or less, or 5 or less amino acid mutations. In general, it is preferred that a substitute amino acid has properties similar to those of the original amino acid in order to conserve protein function. Such amino acid substitution is referred to as “conservative substitution”. For example, Ala, Val, Leu, Ile, Pro, Met, Phe, and Trp are grouped into a class of non-polar amino acids, and have properties similar to one another. Uncharged amino acids include Gly, Ser, Thr, Cys, Tyr, Asn, and Gln. Acidic amino acids include Asp and Glu. Basic amino acids include Lys, Arg, and His. Amino acid substitutions within each group are acceptable.

Herein, the phrase “functionally equivalent to the protein comprising the amino acid sequence of SEQ ID NO: 2” means that the protein has the physicochemical properties (1) to (5) described above.

Those skilled in the art can obtain a polynucleotide encoding a homologue of the α-ketoamide reductase by appropriately introducing substitutional, deletional, insertional, and/or additional mutations into the DNA of SEQ ID NO: 1 by site-directed mutagenesis (Nucleic Acid Res. 10, pp. 6487 (1982); Methods in Enzymol. 100, pp. 448 (1983); Molecular Cloning 2nd Edt., Cold Spring Harbor Laboratory Press (1989); PCR A Practical Approach IRL Press pp. 200 (1991)) or the like. A homologue of α-ketoamide reductase of SEQ ID NO: 2 can be obtained by introducing and expressing the polynucleotide encoding the α-ketoamide reductase homologue within a host.

The α-ketoamide reductase homologue of the present invention refers to a protein exhibiting a homology of at least 50%, preferably at least 70%, more preferably 80%, much more preferably 90% homology, even more preferably 95%, most preferably 98% or higher to the amino acid sequence of SEQ ID NO: 2. Protein homology searches can be performed against protein databases (amino acid sequences), such as SWISS-PROT, PIR, and DAD; DNA sequence databases, such as DDBJ, EMBL, and GenBank; and databases of amino acid sequences deduced from DNA sequences by using programs such as BLAST and FASTA via the Internet.

BLAST homology searches for the amino acid sequence of SEQ ID NO: 2 of this invention gave the highest homology of 47% to Candida parapsilosis-derived conjugated polyketone reductase (conjugated polyketone reductase C2 protein). However, there is no previous report suggesting that the conjugated polyketone reductase has the activity of reducing α-ketoamide.

An α-ketoamide reductase of the present invention may comprise some additional amino acid sequences, as long as it has activity that is functionally equivalent to that of the protein comprising the amino acid sequence of SEQ ID NO: 2. For example, the enzyme may contain tag sequences, such as a histidine tag and a HA tag, or alternatively, the enzyme may be fused to another protein. The carbonyl reductases of the present invention or their homologues may exist as polypeptide fragments, as long as they have activity which is functionally equivalent to that of the protein comprising the amino acid sequence of SEQ ID NO: 2.

The polynucleotides encoding the α-ketoamide reductases of the present invention can be isolated by the methods described below. For example, PCR can be performed to obtain the DNAs of the present invention, using PCR primers based on the nucleotide sequence of SEQ ID NO: 1, and either chromosomal DNA from a strain producing the enzyme, or a cDNA library, as a template. Additionally, the polynucleotides of the present invention can be obtained by digesting chromosomal DNA from a strain producing the enzyme with restriction enzymes, and then inserting these fragments into a phage, plasmid, or the like; and transforming E. coli with the DNA construct. The resulting library, or a cDNA library, can be screened by colony hybridization, plaque hybridization, or similar methods, using the DNA fragment obtained as described above as a probe.

Alternatively, the polynucleotides of the present invention can be obtained by analyzing the nucleotide sequence of the DNA fragment yielded by PCR, and then designing PCR primers for sequence extension based on the sequence obtained. Next, chromosomal DNA of the enzyme-producing strain is digested with appropriate restriction enzymes, and is then subjected to self-circle formation to serve as a template for inverse PCR (Genetics 120, 621-623 (1988)), or alternatively by RACE (Rapid Amplification of cDNA End; “Experimental manual for PCR” p. 25-33, HBJ Press), and such.

The polynucleotides of the present invention include synthetic DNAs in addition to genomic DNAs and cDNAs cloned by the method described above.

Hybridization is carried out as follows. A nucleic acid strand (DNA or RNA) that comprises a sequence complementary to the sequence of SEQ ID NO: 1, or a partial sequence thereof, is used as a probe and is hybridized to a nucleic acid of interest. Then, it is verified whether the nucleic acid significantly hybridizes to the probe after washing under stringent conditions. The probe to be used comprises, for example, 20 consecutive nucleotides or more, preferably 25 nucleotides or more, more preferably 30 nucleotides or more, still more preferably 40 nucleotides or more, yet more preferably 80 nucleotides or more, and yet more preferably 100 nucleotides or more (for example, the full-length sequence of SEQ ID NO: 1). When the probe contains a sequence which is unrelated to the sequence of SEQ ID NO: 1 or the complementary sequence (e.g., a sequence derived from a vector) , a negative control hybridization may be carried out using the unrelated sequence alone as a probe by the same procedure, to confirm that this probe does not significantly hybridize to the sequence of interest after washing under the same conditions. Such hybridizations can be carried out by conventional methods using nitrocellulose membrane, nylon membrane, or similar (Sambrook et al. (1989) Molecular Cloning, Cold Spring Harbor Laboratories; Ausubel, F. M., et al., (eds) (1991) Current Protocols in Molecular Biology, Wiley Interscience, New York).

A specific example of stringent hybridization conditions comprises: overnight hybridization using a solution containing 6×SSC, 0.5% (W/V) SDS, 100 μg/ml denatured salmon sperm DNA, and 5× Denhardt's solution (1× Denhardt's solution contains 0.2% polyvinylpyrrolidone, 0.2% bovine serum albumin, and 0.2% Ficoll) at 45° C., preferably at 55° C., more preferably at 60° C., still more preferably at 65° C.; followed by a post-hybridization wash comprising washing three times with 4×SSC containing 0.5% SDS for 20 minutes at the same temperature as in the hybridization. More preferably, post-hybridization wash comprises washing twice with 4×SSC containing 0.5% SDS for 20 minutes and washing once with 2×SSC containing 0.5% SDS for 20 minutes at the same temperature as in the hybridization. More preferably, post-hybridization wash comprises washing twice with 4×SSC containing 0.5% SDS for 20 minutes and then washing once with 1×SSC containing 0.5% SDS for 20 minutes at the same temperature as in the hybridization. More preferably, post-hybridization wash comprises washing once with 2×SSC containing 0.5% SDS for 20 minutes, washing once with 1×SSC containing 0.5% SDS for 20 minutes, and washing once with 0.5×SSC containing 0.5% SDS for 20 minutes at the same temperature as in the hybridization. More preferably, post-hybridization wash comprises washing once with 2×SSC containing 0.5% SDS for 20 minutes, washing once with 1×SSC containing 0.5% SDS for 20 minutes, washing once with 0.5×SSC containing 0.5% SDS for 20 minutes, and washing once with 0.1×SSC containing 0.5% SDS for 20 minutes at the same temperature as in the hybridization.

The present invention also provides polynucleotides isolated by the method described above. Expression vectors for the α-ketoamide reductase are constructed by inserting a polynucleotide encoding the α-ketoamide reductase of the present invention into a conventional expression vector.

The present invention provides methods to obtain recombinant α-ketoamide reductase of the present invention by culturing cells transformed with the expression vector. The present invention also provides a method for producing the proteins of the present invention with α-ketoamide reducing activity, comprising the step of culturing cells transformed with a recombinant vector containing a polynucleotide of the present invention.

There is no limitation on the type of microorganism to be transformed for the expression of the α-ketoamide reductase of the present invention, as long as it is capable of being transformed with a recombinant vector that contains a polynucleotide encoding a polypeptide encoding the α-ketoamide reductase and is competent to express α-ketoamide reductase activity. The available microorganisms include, the microorganisms listed below.
The genus Escherichia
The genus Bacillus
The genus Pseudomonas
The genus Serratia
The genus Brevibacterium
The genus Corynebacterium
The genus Streptococcus
Bacterial strains, such as those belonging to the genus Lactobacillus, for which host-vector systems are available
The genus Rhodococcus
Actinomycetes, such as those belonging to the genus Streptomyces, for which host-vector systems are available
The genus Saccharomyces
The genus Kluyveromyce
The genus Schizosaccharomyces
The genus Zygosaccharomyces
The genus Yarrowia
The genus Trichosporon
The genus Rhodosporidium
The genus Pichia
Yeasts, such as those belonging to the genus Candida, for which host-vector systems are available
The genus Neurospora
The genus Aspergillus
The genus Cephalosporium
Molds, such as those belonging to the genus Trichoderma, for which host-vector systems are available

Preparation of transformants and construction of recombinant vectors compatible to each host can be achieved by using conventional techniques of molecular biology, bioengineering, and genetic engineering (for example, Sambrook et al., Molecular Cloning, Cold Spring Harbor Laboratories) . To obtain expression of the genes of the present invention for carbonyl reductases that use NADPH as an electron donor in a microorganism, the DNA should be first inserted into a plasmid or phage vector that is stable in the microorganism and which will permit transcription and translation of the genetic information.

To achieve this, a promoter, which is a regulatory unit for transcription and translation, is inserted at the 5′-side (upstream) of the DNA chain of the present invention, and more preferably a terminator is also inserted at the 3′-side (downstream) of the DNA. Such promoters and terminators should be known to be functional in the host microorganisms. Such vectors, promoters, terminators, and others, which can be used with various microorganisms, are described in detail in “Fundamental Microbiology (Biseibutsugaku Kiso-kouza) 8: Genetic Engineering, KYORITSU SHUPPAN CO., LTD.”, and in particular those to be used with yeasts are described in detail in Adv. Biochem. Eng. 43, 75-102 (1990), Yeast 8, 423-488 (1992), and others.

For example, for bacterial species belonging to the genus Escherichia, in particular for Escherichia coli, pBR and pUC plasmids are available as plasmid vectors, and available promoters include those derived from lac (β-galactosidase), trp (tryptophan operon), tac, trc (fusion of lac and trp), and λ phage PL and PR. Available terminators include those derived from trpA, phage, and rrnB ribosomal RNA. Suitable vectors include pSE420D (described in JP-A 2000-189170), which has a modified cloning site derived from the multi-cloning site of the commercially available pSE420 (Invitrogen).

For bacterial species belonging to the genus Bacillus, available vectors include pUB110 plasmids and pC194 plasmids. The plasmids can be integrated into the chromosome. Promoters and terminators are available from genes including apr (alkaline protease), npr (neutral protease), amy (α-amylase).

For Pseudomonas putida, Pseudomonas cepacia, and other species from the genus Pseudomonas, host-vector systems have been developed previously. Wide host-range vectors (containing genes required for autonomous replication derived from RSF1010 and others), such as pKT240, are available; these vectors were constructed from the plasmid TOL, which is involved in the degradation of toluene and toluene-containing compounds. The gene for lipase (JP-A Hei 5-284973) can provide the promoter and terminator.

For bacterial species belonging to the genus Brevibacterium, particularly Brevibacterium lactofermentum, available plasmid vectors include pAJ43 (Gene 39, 281 (1985)). This species can utilize promoters and terminators that are suitable for E. coli, without modification.

For bacterial species belonging to the genus Corynebacterium, particularly Corynebacterium glutamicum, plasmid vectors, such as pCS11 (JP-A Sho 57-183799) and pCB101 (Mol. Gen. Genet. 196, 175 (1984)), are available.

For bacterial species belonging to the genus Streptococcus, plasmid vectors, such as pHV1301 (FEMS Microbiol. Lett. 26, 239 (1985)) and pGK1 (Appl. Environ. Microbiol. 50, 94 (1985)), are available.

For bacterial species belonging to the genus Lactobacillus, the plasmid vector pAMβ1 (J. Bacteriol. 137, 614 (1979)) is available; this plasmid vector was constructed for bacterial species belonging to the genus Streptococcus, and promoters which are suitable for E. coli can also be used.

For bacterial species belonging to the genus Rhodococcus, plasmid vectors isolated from Rhodococcus rhodochrous (J. Gen. Microbiol. 138, 1003 (1992)) are available.

For bacterial species belonging to the genus Streptomyces, plasmids can be constructed by the method of Hopwood et al., which is described in Genetic Manipulation of Streptomyces: A Laboratory Manual Cold Spring Harbor Laboratories (1985). In particular for Streptomyces lividans, available plasmid vectors are: pIJ486 (Mol. Gen. Genet. 203, 468-478 (1986)), pKC1064 (Gene 103, 97-99 (1991)), and PUWL-KS (Gene 165, 149-150 (1995)). Furthermore, the same plasmids can be used for Streptomyces virginiae (Actinomycetol. 11, 46-53 (1997)).

For fungal species belonging to the genus Saccharomyces, particularly Saccharomyces cerevisiae, YRp plasmids, YEp plasmids, YCp plasmids, and YIp plasmids are available. Integration vectors (e.g., EP 537456) which use homologous recombination with multicopy ribosomal DNA on the chromosome, are highly advantageous since a gene of interest can be introduced in multiple copies using the vector, and the integrated gene is stable in the host. Available promoters and terminators are derived from ADH (alcohol dehydrogenase), GAPDH (glyceraldehyde-3-phosphate dehydrogenase), PHO (acid phosphatase), GAL (β-galactosidase), PGK (phosphoglycerate kinase), ENO (enolase), and others.

For fungal species belonging to the genus Kluyveromyces, particularly Kluyveromyces lactis, available plasmid vectors include Saccharomyces cerevisiae-derived 2μm plasmids and pKD1 plasmids (J. Bacteriol. 145, 382-390 (1981)), plasmids derived from pGK11 involved in killer activity, KARS plasmids derived from genes associated with the autonomous replication in fungal species belonging to the genus Kluyveromyces, and vector plasmids capable of integrating into the chromosome via homologous recombination with ribosomal DNA (e.g., EP 537456). Promoters and terminators derived from ADH, PGK, and other genes are also available.

For fungal species belonging to the genus Schizosaccharomyces, available plasmid vectors are: ARS derived from Schizosaccharomyces pombe (genes involved in the autonomous replication) and Saccharomyces cerevisiae-derived plasmid vectors containing selection markers which complement nutritional requirement (Mol. Cell. Biol. 6, 80 (1986)). In addition, the ADH promoter and others derived from Schizosaccharomyces pombe are available (EMBO J. 6, 729 (1987)). In particular, the commercially available pAUR224 (Takara Shuzo) can be readily used.

For fungal species belonging to the genus Zygosaccharomyces, plasmid vectors derived from the Zygosaccharomyces rouxii-derived plasmid pSB3 (Nucleic Acids Res. 13, 4267 (1985)) or the like are available, and available promoters include the PHO5 promoter derived from Saccharomyces cerevisiae and the GAP-Zr (glyceraldehyde-3-phosphate dehydrogenase) promoter derived from Zygosaccharomyces rouxii (Agri. Biol. Chem. 54, 2521 (1990)).

For Pichia angusta (previous name: Hansenula polymorpha) belonging to the genus Pichia, there are previously developed host-vector systems. Pichia angusta-derived genes involved in the autonomous replication (HARS1 and HARS2) can be used as vectors. However, these genes are relatively unstable, and therefore, multicopy integration into the chromosome (Yeast 7, 431-443 (1991)) may be used more advantageously. The AOX (alcohol oxidase) promoter, which is inducible with methanol or the like, or the FDH (formate dehydrogenase) promoter can be used for this fungal species. In addition, for Pichia pastoris and others, host-vector systems using Pichia-derived genes involved in the autonomous replication (PARS1 and PARS2) or the like have been developed (Mol. Cell. Biol. 5, 3376 (1985)). Thus, it is possible to use high efficiency promoters, such as AOX which is inducible with high density culture and methanol (Nucleic Acids Res. 15, 3859 (1987)).

For Candida maltosa, Candida albicans, Candida tropicalis, Candida utilis, and other species belonging to the genus Candida, host-vector systems have been developed. Candida maltosa-derived ARS has been cloned previously (Agri. Biol. Chem. 51, 51, 1587 (1987)), and thus vectors using this ARS have been developed for Candida maltosa. In addition, chromosome integration vectors with high efficiency promoters have been developed for Candida utilis (JP-A Hei 08-173170).

The fungal species Aspergillus niger and Aspergillus oryzae, belonging to the genus Aspergillus, have been extensively studied. Plasmids, chromosome integration vectors, and extracellular protease and amylase derived promoters are also available for the fungal species (Trends in Biotechnology 7, 283-287 (1989)).

For Trichoderma reesei, belonging to the genus Trichoderma, a host-vector system has been developed, and an extracellular cellulase gene-derived promoter and others are available (Biotechnology 7, 596-603 (1989)).

Furthermore, various host-vector systems have been developed for plant and animal species, in addition to microorganisms. Large-scale systems for the expression of foreign proteins in insects, particularly in silkworms (Nature 315, 592-594 (1985)), and plants, such as colza, maize, and potato, have been developed, and are suitable for use.

The present invention also relates to methods for producing optically active alcohols, in particular (R)-α-hydroxy amides and (R)-α-hydroxyesters via the reduction of ketones using the α-ketoamide reductases described above.

Preferred ketones to be used in the methods of the present invention for producing optically active alcohols include α-ketoamides and α-ketoesters. optically active alcohols can be produced using such compounds as substrates. α-Ketoamides to be used in the methods of the present invention for producing optically active alcohols include benzoyl formamide derivatives which may have lower alkyl group, halogen group, nitro group, alkoxy group, hydroxyl group, or the like in the benzene ring, for example, benzoyl formamide, 2-chlorobenzoyl formamide, 3-chlorobenzoyl formamide, and 4-chlorobenzoyl formamide. The α-ketoamides also include α-ketoalkyl amide which may contain an aromatic group, a halogen group, a nitro group, an alkoxy group, a lower alkyl group, or the like, as a substituent, for example, 2-oxopropionamide, 2-oxobutylamide, 2-oxoisobutylamide, 2-oxovaleramide, 2-oxoisovaleramide, 2-oxocaproylamide, and 2-oxoisocaproylamide. The α-ketoesters include α-ketoesters yielded by substituting lower alkyl ester for the amide group of the α-ketoamides described above, for example, 2-oxoisovalerate ethyl ester. The α-ketoesters also include pyruvate ethyl ester, 2-oxoethyl butyrate, ethyl 2-oxovalerate, ethyl 2-oxocaproate, ethyl 2-oxoheptanoate, ethyl 2-oxoisovalerate, ethyl 2-chloroacetoacetate, phenylglyoxal, cyclohexane-1,2-dione, and α-ketopantoyl lactone. The incubation of each of the α-ketoamides and α-ketoesters with the enzyme of the present invention yields a corresponding (R)-α-hydroxy amide or ester. α-Ketoamide derivatives can be produced, for example, by the method described below.

Cuprous cyanide is suspended in a solvent, such as toluene or acetonitrile. A corresponding acid chloride derivative is added dropwise while the suspension is being stirred. After heat reflux, the mixture is cooled down to room temperature. Then, insoluble material is removed by filtration. The solvent is distilled off under reduced pressure. The resulting residue is the carbonyl cyanide derivative, which is then suspended in concentrated hydrochloric acid. The suspension is stirred at room temperature overnight. Then, the whole reaction solution is added to water, and the mixture is stirred. The resulting crystals are separated by filtration, and washed and dried to give an α-ketoamide derivative of interest. Alternatively, the α-ketoamide derivative can be synthesized from the corresponding formic acid derivative as starting material, by chlorinating the carbonyl with thionyl chloride or such, or by reacting it with ammonia via esterification with a short chain alcohol.

A ketone, for example, 2-chlorobenzoyl formamide, which is a reaction substrate used in the present invention, can be used at an appropriate concentration within a range in which the substrate will not inhibit the reaction, so that the product of interest is generated efficiently. The concentration of the 2-chlorobenzoyl formamide substrate in the reaction solution ranges, for example, from 0.01 to 50%, preferably from 0.1 to 20%, more preferably from 0.1 to 10%. There is no limitation on the type of method used to add the substrate. For example, the substrate may be added by the method described in Example 8. The substrate can be added all at once at the start of reaction, but it is preferable to add the substrate continuously or stepwise so that the substrate concentration does not become too high in the reaction solution. The reaction may be continued typically for one to five days, preferably for one to three days. The reaction temperature may range from 4 to 60° C., preferably from 15 to 37° C. The pH during the reaction may range from 3 to 11, preferably from 5 to 9.

The optically active alcohols produced by the methods of the present invention include (R)-α-hydroxy amides and (R)-α-hydroxyesters, more preferably (R)-mandelamide derivatives, still more preferably, (R)-2-chloromandelamide, (R)-3-chloromandelamide, (R)-4-chloromandelamide, (R)-mandelamide, (R)-ethyl lactate, ethyl (R)-2-hydroxybutyrate, ethyl (R)-2-hydroxy valerate, ethyl (R)-2-hydroxy caproate, ethyl (R)-2-hydroxy heptanoate, ethyl (R)-2-hydroxyisovalerate, ethyl (S)-4-chloro-3-hydroxybutyrate, (R)-mandelaldehyde, (R)-2-hydroxy cyclohexanone, and D-pantoyl lactone.

Herein, the term “optically active alcohol” refers to an alcohol in which the amount of one optical isomer is larger than that of the other optical isomer, or an alcohol comprising either optical isomer. The “optical isomer” of the present invention is sometimes called “optically active substance” or “enantiomer”.

Optically active alcohols yielded can be purified using an appropriate combination of: centrifugal separation of microbial cells and proteins, separation with membrane treatment, solvent extraction, distillation, crystallization, and others.

For example, microbial cells are removed from a reaction solution containing (R)-2-chloromandelamide by centrifugation, and proteins are removed by ultrafiltration. Then, (R)-2-chloromandelamide is extracted using ethyl acetate or the like, and the solvent is distilled off under reduced pressure. The optically active alcohols can be purified by the procedure described above.

The optical purity (enantiomeric excess; ee) of an optically active alcohol produced by the method of the present invention is preferably 80% ee or higher, more preferably 90% ee or higher, still more preferably 99% ee or higher. The optical purity of the product can be estimated by analyzing the reaction product with an optical resolution column or the like.

Alternatively, an optically active alcohol can be produced via a desired enzymatic reaction, by contacting the reaction solution with transformed microbial cells that express the functional enzyme of the present invention, for example. The procedure of contacting the enzyme with the reaction solution is not limited to the specific examples described above. Microorganisms to be used in the method include heterologous transformants expressing the functional protein according to SEQ ID NO: 2, for example, E. coli transformed with pSE-YDL1 or pSG-YDL1 for co-expression of an enzyme catalyzing the regeneration of coenzyme NADPH, for example, glucose dehydrogenase.

Processed products of transformants containing the α-ketoamide reductase of the present invention specifically include microorganisms treated with a detergent or an organic solvent (such as toluene) to alter the membrane permeability, dried microbial cells prepared by freeze-drying or spray-drying, cell-free extracts prepared by lysing microbial cells by treatment with glass beads or an enzyme, partially purified material from the extract, purified enzyme, and immobilized enzyme or microorganisms prepared by immobilizing enzyme or transformants.

In a preferred embodiment, the present invention provides methods for producing (R)-2-chloromandelamide, which comprise incubating 2-chlorobenzoylformamide with the α-ketoamide reductase of the present invention, transformants expressing the α-ketoamide reductase, or the processed products of the transformants.

The regeneration of NADPH from NADP+ (that is produced from NADPH in the reduction described above) can be achieved by using the ability of a microorganism to reduce NADP+ (glycolysis pathway, assimilation pathway for methylotroph C1 compound, etc.). The ability to reduce NADP+ can be potentiated by adding glucose, ethanol, or the like to the reaction system. The reduction reaction can be achieved by the addition of either a microorganism with the ability to reduce NADP+ to NADPH, or a processed product or enzyme thereof, to the reaction system. For example, the regeneration of NADPH can be achieved using a microorganism containing glucose dehydrogenase, alcohol dehydrogenase, formate dehydrogenase, amino acid dehydrogenase, or organic acid dehydrogenase (malate dehydrogenase), or a processed product thereof, or a purified or partially purified enzyme. Such reaction components required for NADPH regeneration may either be added to the reaction system to produce an optically active alcohol according to the present invention, added to the system after being immobilized, or can be contacted with the system via NADPH-permeable membrane.

Under some circumstances in the method, when live microbial cells transformed with a recombinant vector containing a polynucleotide of the present invention are used to produce the optically active alcohols described above, no additional reaction system is required for the subsequent NADPH regeneration step. Specifically, an efficient reduction can be achieved when the transformed microbial host has a high enough NADPH regeneration activity, without adding the enzyme for NADPH regeneration. Furthermore, the expression of NADPH regeneration enzyme and NADPH dependent α-ketoamide reductases, as well as the reduction reaction, can be achieved more efficiently when a host is co-transformed with a DNA encoding the NADPH-dependent α-ketoamide reductase of the present invention and a gene encoding a NADPH regeneration enzyme of glucose dehydrogenase, alcohol dehydrogenase, formate dehydrogenase, amino acid dehydrogenase, organic acid dehydrogenase (e.g., malate dehydrogenase) , or such. Two or more of the genes described above can be introduced into a host without plasmid incompatibility, by either: inserting the genes independently into multiple vectors with distinct replication origins, followed by the transformation of the host with the recombinant vectors; by introducing both genes into a single vector; or by integrating both or either of the genes into the chromosome.

When a plurality of genes are inserted into a single vector, regulatory regions for expression, such as the promoter and terminator, maybe linked to each gene, or alternatively the genes may be expressed as an operon containing multiple cistrons, such as lactose operon.

Glucose dehydrogenases derived from the species belonging to the genus Bacillus, the genus Pseudomonas, the genus Thermoplasma, and others, can be used as the NADPH regeneration enzymes. Specifically, preferred recombinant vectors include pSG-YDL1, which contains the genes encoding the α-ketoamide reductase and the glucose dehydrogenase derived from Bacillus subtilis as inserts.

The reduction by the enzyme of the present invention can be carried out using water; an organic solvent immiscible with water, for example, ethyl acetate, butyl acetate, toluene, chloroform, n-hexane, methyl isobutyl ketone, methyl-tert-butyl ester; or a mixed solvent system, such as a two-phase system comprising an aqueous medium and an organic solvent miscible with water, for example, methanol, ethanol, isopropyl alcohol, acetonitrile, acetone, or dimethylsulfoxide. The reaction of the present invention can also be achieved using immobilized enzymes, membrane reactors, and other methods.

If required, the coenzymes NADP+ or NADPH may be added to the reaction system at a concentration of 0.001 mM to 100 mM, preferably 0.01 to 10 mM.

To regenerate NADPH, for example, glucose may be added to the reaction system when glucose dehydrogenase is used, or ethanol or isopropanol may be added to the system when alcohol dehydrogenase is used. Such a compound may be added to the substrate ketone at a molar ratio of 0.1 to 20, preferably 1 to 5. On the other hand, the NADPH regeneration enzyme, such as glucose dehydrogenase or alcohol dehydrogenase, which has enzymatic activity 0.1 to 100 times higher, preferably about 0.5 to 20 times higher than that of the NADPH-dependent α-ketoamide reductase of the present invention may also be used.

Optically active alcohols produced by the reduction of ketones according to the present invention can be purified using an appropriate combination of centrifugal separation of microbial cells and proteins, separation with membrane treatment, solvent extraction, distillation, crystallization, and others.

For example, (R)-2-chloromandelamide can be obtained as an optically active alcohol by the centrifugal removal of microbial cells from the reaction solution containing transformant, followed by extraction of the solution with ethyl acetate, butyl acetate, toluene, hexane, benzene, methyl isobutyl ketone, methyl-tert-butyl ether, butanol, or the like, and concentration under reduced pressure. For higher purity, the reaction product may be recrystallized from an appropriate solvent, or fractionated by silica gel column chromatography. Such treatments ensure higher purity of the product.

The present invention provides NADPH-dependent α-ketoamide reductases that can be used to produce optically active alcohols, methods for producing the enzymes, and efficient methods using transformants for producing high optical purity (R)-2-chloromandelamide.

(R)-2-chloromandelamide is a compound that can, be used for various pharmaceutical products. In addition, α-hydroxy amides, such as (R)-2-chloromandelamide and (R)-3-chloromandelamide, are reduced readily by a reducing agent, such as lithium aluminium hydride or boron, to synthesize α-hydroxy amines, such as (R)-2-amino-1-(2-chlorophenyl) ethanol and (R)-2-amino-1-(3-chlorophenyl) ethanol, that can also be used for various pharmaceutical products.

Any patents, patent applications, and publications cited herein are incorporated by reference.

### EXAMPLES

The present invention is illustrated in detail below with reference to Examples, but is not to be construed as being limited thereto.

### Example 1

### Purification of α-ketoamide Reductase

500 g of live baker's yeast was purchased from Oriental yeast and the fungal cells were used to purify the enzyme. 500 g of live baker's yeast was suspended in 500 mL of a fungal cell lysis buffer (10 mM potassium phosphate buffer (pH 8.0), 0.02% 2-mercaptoethanol, 1 μM pepstatin A, 1 μM leupeptin, and 1 mM phenylmethanesulfonyl fluoride), and then crushed in a Mini-Lab (Raney). Fungal cell debris was removed by centrifugation to obtain a cell-free extract. Protamine sulfate was added to the cell-free extract, and nucleic acids were removed by centrifugation. Ammonium sulfate was added to the resulting supernatant to obtain 70% saturation. The enzyme was obtained from the resulting precipitated fraction, which was collected by centrifugation.

The resulting precipitate was dissolved in 150 mL of 100 mM potassium phosphate buffer (pH 7.0), and dialyzed against buffer A (10 mM potassium phosphate buffer (pH 7.0), 1 mM ethylenediamine tetraacetic acid (hereinafter abbreviated as EDTA), and 1 mM dithiothreitol (hereinafter abbreviated as DTT)). The resulting precipitate was removed by centrifugation. The enzyme was adsorbed to a DEAE-TOYOPEARL 650S (2.6×19 cm; Tosoh) column pre-equilibrated with the same buffer A. The enzyme was eluted with a concentration gradient of 0-0.4 M potassium chloride. The active fractions were concentrated by ultrafiltration, and then dialyzed against buffer A.

The dialyzed enzyme was adsorbed to MonoQ (1.0×1.0 cm; Amersham Biosciences) pre-equilibrated with the same buffer. After washing with the same buffer, the enzyme was eluted with a concentration gradient of 0-0.2 M potassium chloride. Each fraction was assayed for 2-chlorobenzoyl formamide reducing ability to identify active fractions.

Ammonium sulfate was added to the enzyme-active fractions to obtain 45% saturation. The enzyme was adsorbed to Phenyl-Superose (1.0×1.0 cm, Amersham Biosciences) pre-equilibrated with buffer B (50 mM potassium phosphate buffer (pH 7.0), 1 mM EDTA, and 1 mM DTT), and was then eluted with a concentration gradient of 2.0-0 M ammonium sulfate. Active fractions were then collected. 2CBFD asymmetric reduction was carried out using the active fractions. The product from each fraction was assessed for optical purity. Some fractions were found to give lower optical purity. Thus, only fractions giving high optical purity product were collected and combined together. The mixed sample was used in subsequent experiments.

The active fraction was dialyzed against buffer C (5 mM potassium phosphate buffer (pH 7.0), 1 mM EDTA, and 1 mM DTT), and then adsorbed onto AF-Red-TOYOPEARL 650M column (Tosoh) pre-equilibrated with the same buffer C. The enzyme was eluted with a concentration gradient of 0-1 M potassium chloride, and the active fractions were collected.

The active fractions were concentrated by ultrafiltration, and loaded onto HiLoad 16/60 Superdex 200 (1.0×30 cm, Amersham Biosciences) pre-equilibrated with buffer D (100 mM potassium phosphate buffer (pH 7.0), 0.2 M potassium chloride, 1 mM DTT, and 1 mM EDTA). The samples were fractionated by gel filtration using the same buffer D. The active fractions were collected.

The purified enzyme gave a single band by 12.5% SDS-PAGE. The specific activity of the purified enzyme was 105 U/mg. The purification process is summarized in Table 1.

TABLE 1

| | | | | Specific | |
|---|---|---|---|---|---|
| | Volume | Protein | Total | activity | |
| Step | ml | mg | activity U | U/mg | Yield % |
| Cell-free extract | 1310 | 126000 | 216 | 0.00171 | 100 |
| Protamine sulfate | 1820 | 67700 | 252 | 0.00372 | 117 |
| 70% Ammonium sulfate precipitation | 258 | 4140 | 408 | 0.0986 | 189 |
| DEAE-TOYOPEARL | 140 | 193 | 356 | 1.84 | 165 |
| MonoQ | 50 | 149 | 299 | 2.01 | 138 |
| Phenyl-Superose | 14 | 5.11 | 79.1 | 15.5 | 37 |
| Red-TOYOPEARL | 7.0 | 0.529 | 34.7 | 65.6 | 16 |
| HiLoad 16/60 | 1.5 | 0.185 | 19.4 | 105 | 9 |
| Superdex 200 | | | | | |

### Example 2

### Determination of the Molecular Weight of α-ketoamide Reductase

The molecular weight of a subunit of the enzyme prepared in Example 1 was determined to be approximately 36,000 by SDS-PAGE, and approximately 33,000 using a gel filtration column of HiLoad 16/60 Superdex 200. Thus, the enzyme was deduced to be monomeric.

### Example 3

### Optimal pH for α-ketoamide Reductase

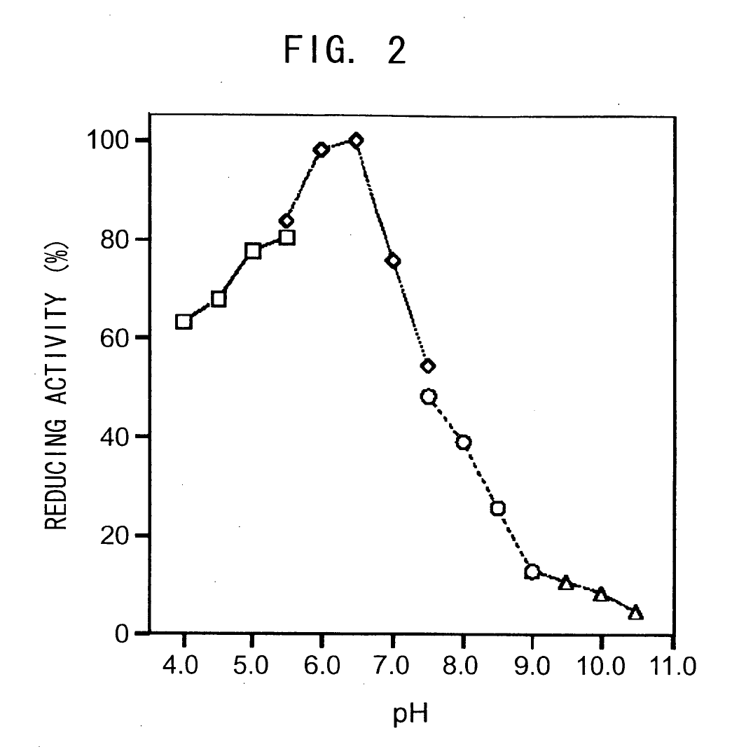

The pH of the reaction solution was altered using 0.1 M sodium acetate buffer (pH 4.0-5.5), potassium phosphate buffer (pH 5.5-7.5), Tris-hydrochloride buffer (pH 7.5-9.0), and glycine-potassium hydroxide buffer (pH 9.0-10.5) to assess the pH dependency of the 2-chlorobenzoyl formamide-reducing activity of the enzyme obtained in Example 1. The activity at each pH was determined as a relative activity when the maximal activity was taken as 100. The result is shown in FIG. 2. The optimal pH (pH range in which the relative activity is 80% or higher) was 5.5-6.5.

### Example 4

### Optimal Temperature for α-ketoamide Reductase

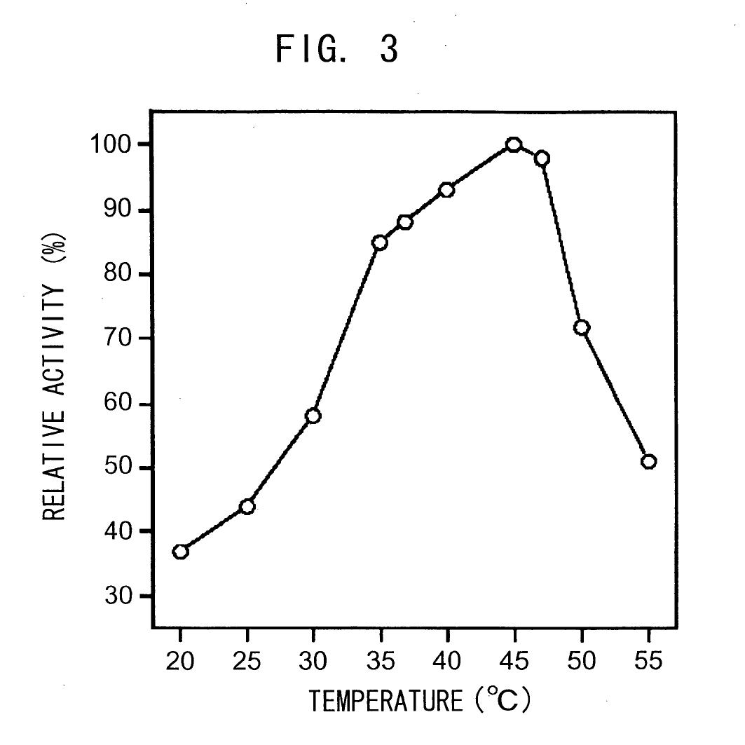

The enzyme obtained in Example 1 was assessed for 2-chlorobenzoyl formamide-reducing activity under standard reaction conditions at various temperatures. The activity at each temperature was determined as a relative activity when the maximal activity was taken as 100. The result is shown in FIG. 3. The optimal temperature (temperature range in which the relative activity is 80% or higher) was 35 to 47° C.

### Example 5

### pH Stability of α-ketoamide Reductase

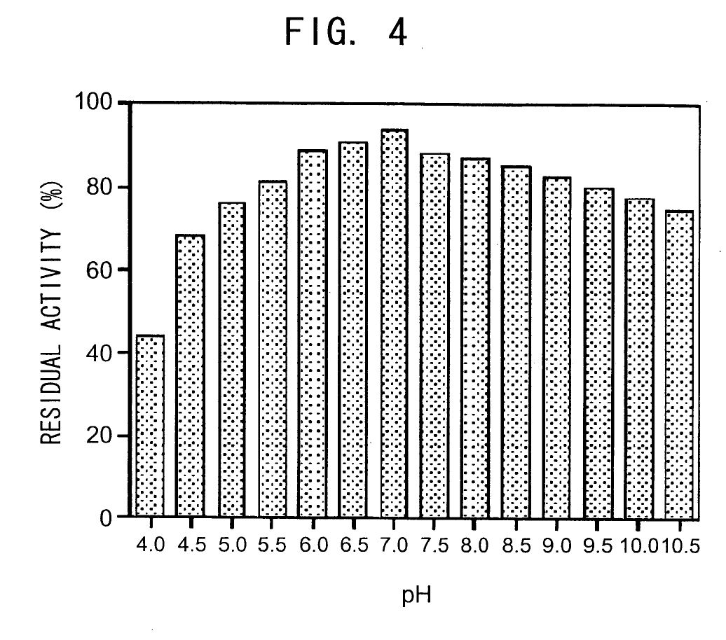

The purified enzyme was incubated in 0.1 M sodium acetate buffer (pH 4.0-5.5) , 0.1 M potassium phosphate buffer (pH 6.0-7.5), 0.1 M Tris-hydrochloride buffer (pH 8.0-9.0), or 0.1 M glycine-potassium hydroxide buffer (pH 9.5-10.5) at 37° C. for 10 minutes. Then, the enzymatic activity was determined as a relative activity when the activity before the treatment was taken as 100. The result is shown in FIG. 4. The stable pH range (pH range in which the residual activity is 80% or higher) was pH 5.5 to 9.5.

### Example 6

### Thermostability of α-ketoamide Reductase

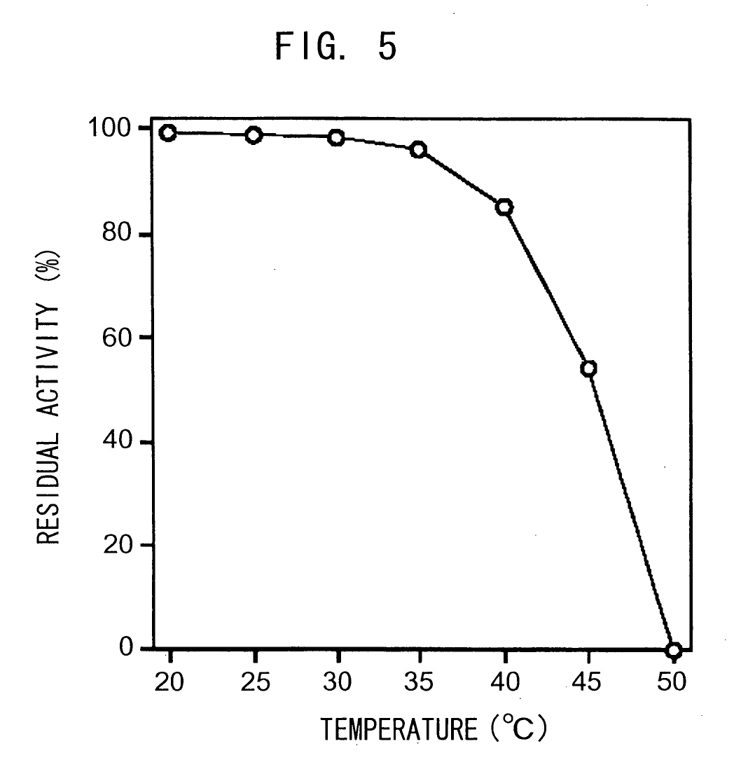

The purified enzyme was incubated in 100 mM potassium phosphate buffer (pH 7.0) at 20 to 50° C. for 30 minutes. The residual activity when the activity before the incubation is taken as 100 is shown in FIG. 5. The temperature range in which the enzyme activity is stable (temperature range in which the residual activity is 80% or higher) falls within the range comprising 40° C. and lower temperatures.

### EXAMPLE 7

### Substrate Specificity of α-ketoamide Reductase

The enzyme obtained in Example 1 was incubated with any one of various α-ketoamides, α-ketoesters, μ-ketoesters, aldehydes, ketones, and others. The reducing activity is represented as a relative activity when the activity of reducing 2-chlorobenzoyl formamide is taken as 100. The result is shown in Table 2.

TABLE 2

| Substrate | Reducing activity (%) |
|---|---|
| 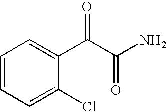 | 100 |
| 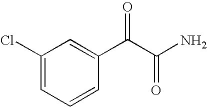 | 143 |
| 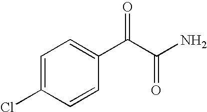 | 71 |
| 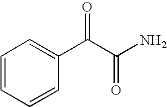 | 59 |
| 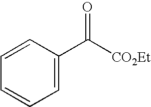 | 32 |
| 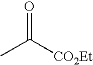 | 9 |
| 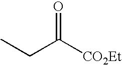 | 33 |
| 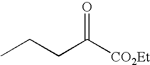 | 46 |
| 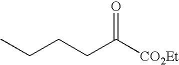 | 49 |
| 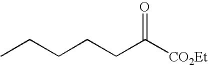 | 50 |
| 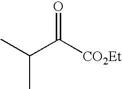 | 71 |
| 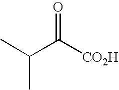 | 0 |
| 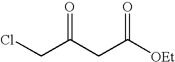 | 4 |
| 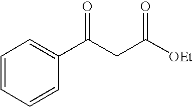 | 0 |
| 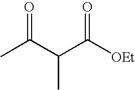 | 0 |
| 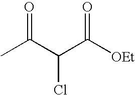 | 0 |
| 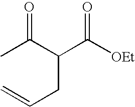 | 0 |
| 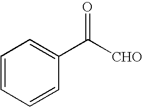 | 120 |
| 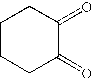 | 10 |
| 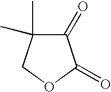 | 19 |
| 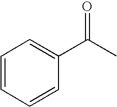 | 0 |
| 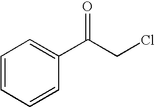 | 0 |
| 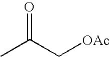 | 0 |
| 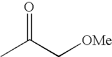 | 0 |

Substrate concentration: 1 mM

### Example 8

### Stereoselectivity of α-ketoamide Reductase

0.2 U of the purified enzyme and each of various α-ketoamides and α-ketoesters (10 μmol each) were incubated in 0.5 ml of 0.1 M potassium phosphate buffer (pH 7.0) in the presence of 10 μmol NADPH at 37° C. for 6 hours. The resulting α-hydroxy amides and α-hydroxyesters were extracted with ether, and then the optical purity of each compound was determined. The result is shown in Table 3.

TABLE 3

| Substrate | Optical purity (%) | Configuration |
|---|---|---|
| 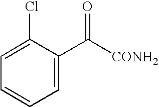 | >99 | R |
| 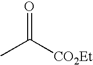 | >99 | R |
| 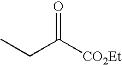 | >99 | R |
| 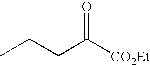 | 98 | R |
| 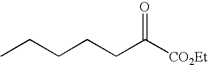 | >99 | R |
| 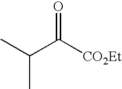 | >99 | R |
| 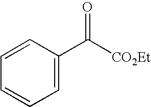 | 97 | R |

The optical purity was determined by the procedure described below.

The optical purity of 2-chloromandelamide was determined by chiral HPLC method or chiral GC method. The chiral HPLC method comprises chromatography using CHIRALPAK AS-H (DAICEL CHEMICAL INDUSTRIES, LTD.). The elution was carried out using a mixed solution of n-hexane and ethanol (85:15) at a flow rate 1.0 mL/min and a column temperature of 25° C. The elution peak for the compound was detected by UV absorbance at 254 nm. Under these conditions, 2-chloromandelamide was eluted 7.5 minutes (R-isomer) and 10.1 minutes (S-isomer) after the start of elution.

The chiral GC method comprises chromatography using GAMMA DEX 225 (SUPELCO; 30 m×0.25 mm×0.25 μm film thickness) under the following conditions: column temperature, 190° C.; injection temperature, 220° C.; detection temperature 220° C.; carrier gas, He/1.0 kg/cm². Under the conditions described above, the S-isomer was eluted 27.0 minutes, and the R-isomer was eluted 29.3 minutes after the start of the elution.

The optical purities of the other α-hydroxyesters were determined by the chiral GC method described below using Chirasil-DEX CB column (Varian; 25 mm×0.25 mm×0.25 μm film thickness). GAMMA DEX 225 was used to determine the optical purities of ethyl lactate (injection temperature, 180° C.; column temperature, 85° C.; detection temperature, 180° C.; R-isomer, 6.13 minutes; S-isomer, 6.82 minutes), ethyl 2-hydroxybutyrate (injection temperature, 180° C.; column temperature, 100° C.; detection temperature, 180° C.; R-isomer, 5.88 minutes; S-isomer, 6.32 minutes), ethyl 2-hydroxyvalerate (injection temperature, 180° C.; column temperature, 110° C.; detection temperature, 180° C.; R-isomer, 6.17 minutes; S-isomer, 6.88 minutes), ethyl 2-hydroxy heptanoate (injection temperature, 180° C.; column temperature, 120° C.; detection temperature, 180° C.; R-isomer, 11.20 minutes; S-isomer, 12.00 minutes), and 2-hydroxyisovalerate (injection temperature, 180° C.; column temperature, 90° C.; detection temperature, 180° C.; R-isomer, 10.42 minutes; S-isomer, 10.89 minutes).

### Example 9

### Partial Amino Acid Sequence of α-ketoamide Reductase

The N-terminal amino acid sequence of the enzyme obtained in Example 1 was analyzed in a protein sequencer. However, no amino acid was detectable. This suggests the possibility that the N-terminus has been blocked. Next, the enzyme was fractionated by 12.5% SDS-PAGE, and a gel piece containing the α-ketoamide reductase was excised from the gel. After washing twice, the protein was treated by in-gel digestion using trypsin at 35° C. overnight. The peptides yielded by the digestion were fractionated by reversed-phase HPLC (Tosoh TSK gel ODS-80Ts; 2.0 mm×250 mm) using a concentration gradient of acetonitrile in 0.1% trifluoroacetic acid.

The peak fraction for a peptide was referred to as T79, and the amino acid sequence was analyzed in a protein sequencer (Hewlett Packard G1005A Protein Sequencer System). The amino acid sequence of T79 is shown in SEQ ID NO: 3.

### Example 10

### Purification of Chromosomal DNA of Saccharomyces cerevisiae

Cells of Saccharomyces cerevisiae ATCC 208277 were cultured in YM medium, and then harvested. Chromosomal DNA was purified from the fungal cells by the method described in Meth. Cell Biol. 29, 39-44 (1975).

### Example 11

### Cloning of α-ketoamide Reductase Gene

Two PCR primers (YDL124w-A1 and YDL124w-T1) were designed based on a deduced open reading frame (YDL124w) according to SEQ ID NO:1, which has been deposited under the accession No. 274172 in DDBJ. The primers are shown in SEQ ID NOs: 4 and 5.

PCR was carried out using GeneAmp PCR System 2400 (Perkin Elmer) A 50 μL reaction solution containing the primers YDL124w-A1 and YDL124w-T1 (10 pmol each), dNTP (10 nmol), chromosomal DNA (200 ng) derived from Saccharomyces cerevisiae, Pyrobest DNA polymerase buffer (Takara Bio), 2 U of Pyrobest DNA polymerase (Takara Bio) underwent 25 cycles of: denaturation (94° C./30 seconds), annealing (55° C. /60 seconds), and extension (72° C./1 minute and 25 seconds). Specific PCR products were amplified.

The amplified product was treated with phenol, and then double-digested with the restriction enzymes AflIII and XbaI. Using a TAKARA Ligation Kit, the resulting DNA fragment was ligated with the vector pSE420D, which had been double-digested with the restriction enzymes NcoI and XbaI. Cells of E. coli DH10B strain were transformed with the ligated DNA, and grown on LB medium containing ampicillin (50 mg/L). Plasmids were purified from the resulting transformants using Miniprep DNA Purification Kit (Takara Bio).

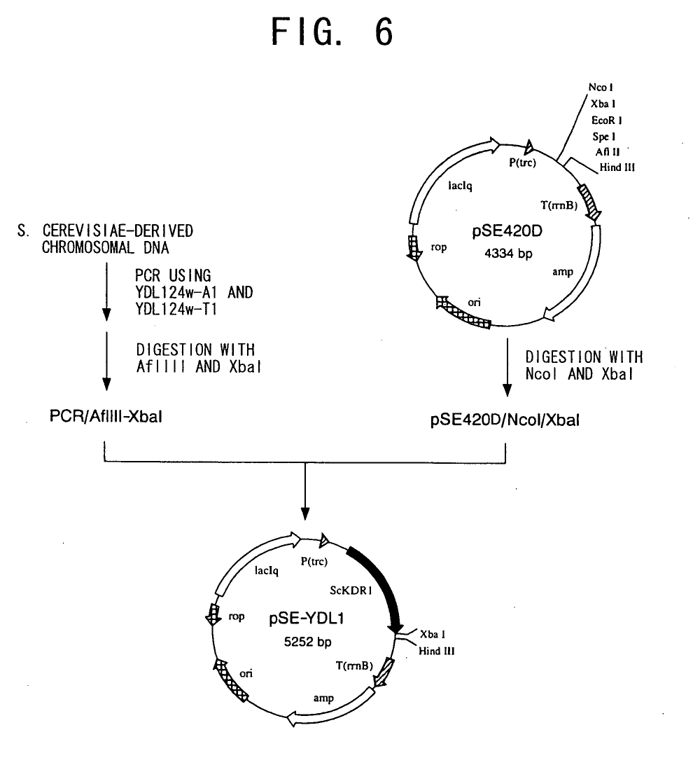

According to the sequencing result, the nucleotide sequence of the inserted DNA fragment was completely identical to the sequence deposited as YDL124w in DDBJ. The plasmid obtained was referred to as pSE-YDL1. The process of plasmid construction is illustrated in FIG. 6.

### Example 12

### Construction of Plasmid pSG-YDL1 for the Co-expression of α-ketoamide Reductase Gene and Glucose Dehydrogenase Gene Derived from Bacillus subtilis

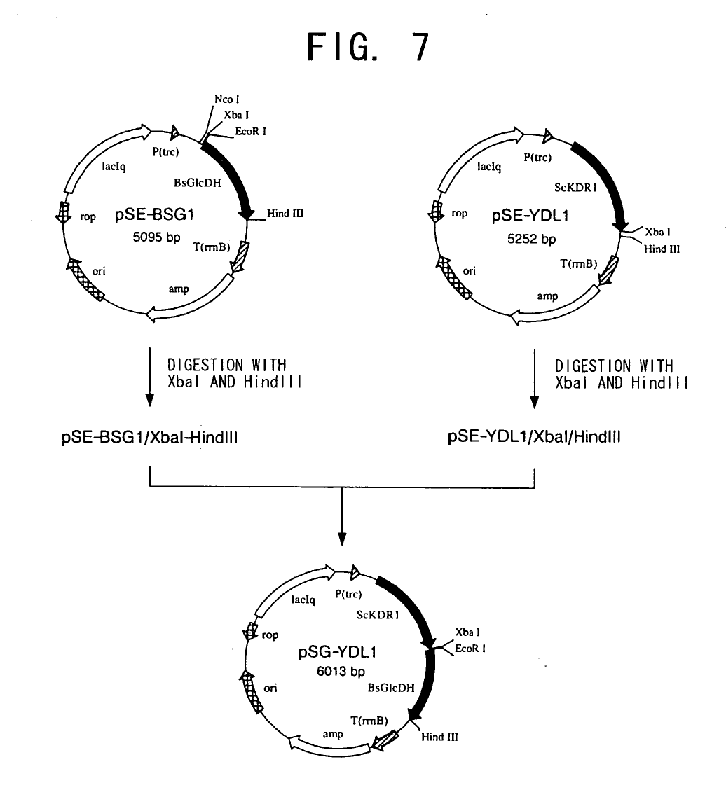

The plasmid pSE-BSG1 (Japanese Patent Application No. 2000-374593) containing the glucose dehydrogenase gene derived from Bacillus subtilis was double-digested with the restriction enzymes XbaI and HindIII to prepare an XbaI-HindIII fragment. pSE-YDL1 was double-digested with the same restriction enzymes. The DNA fragment described above was ligated with the plasmid using Takara Ligation Kit. Cells of E. coli DH10B strain were transformed with the ligated DNA, and then grown on LB medium containing ampicillin (50 mg/L). Plasmids were purified from the resulting transformants using Miniprep DNA Purification Kit (Takara Bio). Thus, the plasmid pSG-YDL1 was constructed, which ensures the co-expression of glucose dehydrogenase and YGL157w. The process of the plasmid construction is illustrated in FIG. 7.

### Example 13

### Assessment for the Activity of YDL124w

Cells of E. coli DH10B strain containing pSE-YDL1 or pSG-YDL1. were grown on LB medium containing ampicillin. Cells were induced with 0.1 mM IPTG for 4 hours, and then the cells were harvested by centrifugation.

Each sample of cells was suspended in cell lysis buffer (50 mM potassium phosphate buffer (pH 8.0) containing 0.02% 2-mercaptoethanol) . The cells were crushed by sonication. Then, the lysates were centrifuged and the resulting supernatants were saved as cell-free extracts.

The 2-chlorobenzoyl formamide-reducing activity was determined to be 1.84 U/mg for pSE-YDL1, and 1.34 U/mg for pSG-YDL1. The 2-chlorobenzoyl formamide-reducing activity was undetectable for the host itself.

In addition, the glucose dehydrogenase activity was determined to be 1.43 U/mg for E. coli DH10B strain containing pSG-YDL1.The glucose dehydrogenase activity was determined by the method described below. The reaction was performed at 30° C. using a reaction solution that contained the enzyme, 100 mM D-glucose, 2.5 mM NAD+, and 100 mM potassium phosphate buffer (pH 6.5). The increase in absorbance at 340 nm which accompanies the formation of NADH was measured. 1U was defined as the amount of enzyme required to catalyze the formation of 1 μmol NADH in one minute.

### Example 14

### Synthesis of (R)-2-chloromandelamide using YDL124w

E. coli cells containing pSG-YDL1 were cultured in 200 mL of 2×YT medium (20 g/L Bacto-peptone, 10 g/L Bacto-yeast extract, and 10 g/L sodium chloride (pH 7.2)). The culture was centrifuged to harvest the bacterial cells. The cells were suspended in 16 mL of cell lysis buffer containing 50 mM potassium phosphate buffer (pH 8.0) and 0.02% 2-mercaptoethanol, and were then crushed by sonication. The bacterial cell lysate was centrifuged to prepare the cell-free extract.

The cell-free extract was assayed for α-ketoamide-reducing activity and glucose dehydrogenase activity. The activities were found to be 2.21 U/mg-protein and 2.81 U/mg-protein, respectively. Ammonium sulfate was added to the cell-free extract at a final concentration of 70% saturation. The mixture was stirred overnight. The resulting precipitate was yielded by centrifugation, and then the precipitated fraction was dissolved in cell lysis buffer and dialyzed against the same buffer. The resulting solution was used in the reaction.

The reaction and analyses were carried out by the same procedures as described in Example 8. As with the purified enzyme, (R)-2-chloromandelamide with 99% ee or higher purity was formed quantitatively from 2-chlorobenzoyl formamide, and ethyl (R)-2-hydroxyisovalerate with 99% ee or higher purity was formed quantitatively from 2-oxo isovalerate ethyl ester. YDL124w was thus confirmed to encode the α-ketoamide reductase.

1. An α-ketoamide reductase comprising the physicochemical properties of:
(1) activity:
reduces a ketone using NADPH as coenzyme to produce an optically active alcohol;
(2) substrate specificity:
(a) uses NADPH as coenzyme in reduction reactions; and
(b) reduces 2-chlorobenzoyl formamide to produce
(R)-2-chloromandelamide with an optical purity of 98% ee or higher;
(3) molecular weight:
approximately 33,000 by gel filtration, and approximately 36,000 by SDS-PAGE;
(4) optimal pH:
pH 5.5 to 6.5; and
(5) optimal temperature:
35 to 47° C.

2. A method for producing an optically active alcohol, wherein the method comprises reacting a ketone with the α-ketoamide reductase according to claim 1, and wherein the produced optically active alcohol corresponds to the ketone.

3. The method for producing an optically active alcohol according to claim 2, wherein the ketone is an α-ketoamide and the corresponding optically active alcohol is an optically active α-hydroxy amide.

4. The method for producing an optically active alcohol according to claim 3, wherein the α-ketoamide is a benzoyl formamide derivative and the corresponding optically active α-hydroxy amide is an (R)-mandelamide derivative.

5. A method for producing an optically active alcohol, wherein the method comprises reacting a ketone with a transformant expressing an α-ketoamide reductase encoded by a polynucleotide according to any one of (a) to (e), or a processed product thereof,
(a) a polynucleotide comprising the nucleotide sequence of SEQ ID NO: 1;
(b) a polynucleotide encoding a protein comprising the amino acid sequence of SEQ ID NO: 2;
(c) a polynucleotide encoding a protein comprising the amino acid sequence of SEQ ID NO: 2, wherein one or more amino acids have been substituted, deleted, inserted, and/or added;
(d) a polynucleotide that hybridizes under stringent conditions to a DNA comprising the nucleotide sequence of SEQ ID NO: 1; and
(e) a polynucleotide encoding an amino acid sequence exhibiting a homology of 50% or higher to the amino acid sequence of SEQ ID NO: 2,
and wherein the produced optically active alcohol corresponds to the ketone.

6. The method for producing an optically active alcohol according to claim 5, wherein the ketone is an α-ketoamide or α-ketoester and the corresponding optically active alcohol is an (R) -α-hydroxy amide or (R)-α-hydroxyester.

7. The method for producing an optically active alcohol according to claim 6, wherein the α-ketoamide is a benzoyl formamide derivative and the (R)-α-hydroxy amide is an (R)-mandelamide derivative.

8. A method for producing an α-ketoamide reductase, which comprises the step of culturing a transformant expressing the α-ketoamide reductase according to claim 1, or an α-ketoamide reductase encoded by any one of:
(a) a polynucleotide comprising the nucleotide sequence of SEQ ID NO: 1;
(b) a polynucleotide encoding a protein comprising the amino acid sequence of SEQ ID NO: 2;
(c) a polynucleotide encoding a protein comprising the amino acid sequence of SEQ ID NO: 2, wherein one or more amino acids have been substituted, deleted, inserted, and/or added;
(d) a polynucleotide that hybridizes under stringent conditions to a DNA comprising the nucleotide sequence of SEQ ID NO: 1; and
(e) a polynucleotide encoding an amino acid sequence exhibiting 50% or higher homology to the amino acid sequence of SEQ ID NO: 2.


SEQUENCE LISTING (5 sequences)

SEQ ID NO: 1 - DNA, 939 bp, Saccharomyces cerevisiae
atgtcatttcaccaacagttctttaccttgaataatggaaataaaatccccgcaatcgccatcattgggacaggtactagatggtataaaaacgaagaaacggatgctaccttttcgaacagtttggtcgaacagattgtttatgctctgaagttacctggcattattcacattgatgctgctgagatctacagaacatatccagaagttgggaaggcacttagcctcaccgaaaaaccaagaaatgcaatattcttgacagacaagtactcacctcaaatcaagatgtcagattccccagcggatggactagatttagctttgaagaagatgggcactgactatgtcgatctataccttttgcatagcccatttgtttccaaagaggtcaacgggttaagtttggaggaagcctggaaggacatggagcaattgtacaaatcaggtaaagctaaaaatatcggtgtttccaactttgctgtggaagacttgcaaagaattctgaaagttgcggaagtcaagccccaagttaatcaaattgagttcagtcccttcttgcagaatcaaacaccagggatctacaaattttgccaagaacatgatatattggtagaagcatactcgccactaggtcccttacaaaagaaaacagcacaagatgactctcaaccgtttttcgaatacgtgaaagagttatccgagaaatatatcaaatctgaagcccaaattatcctacgttgggtgaccaaacgtggcgtgttgcctgtaaccacctcttccaaacctcaaagaatttctgacgcgcaaaatttattctctttcgacttgacggctgaggaagtcgataagataacggagttgggcttggaacatgaaccgctaagattgtattggaataaattgtacggtaaatacaactacgctgctcaaaaagtataa

SEQ ID NO: 2 - protein, 312 aa, Saccharomyces cerevisiae
MSFHQQFFTLNNGNKIPAIAIIGTGTRWYKNEETDATFSNSLVEQIVYALKLPGIIHIDAAEIYRTYPEVGKALSLTEKPRNAIFLTDKYSPQIKMSDSPADGLDLALKKMGTDYVDLYLLHSPFVSKEVNGLSLEEAWKDMEQLYKSGKAKNIGVSNFAVEDLQRILKVAEVKPQVNQIEFSPFLQNQTPGIYKFCQEHDILVEAYSPLGPLQKKTAQDDSQPFFEYVKELSEKYIKSEAQIILRWVTKRGVLPVTTSSKPQRISDAQNLFSFDLTAEEVDKITELGLEHEPLRLYWNKLYGKYNYAAQKV

SEQ ID NO: 3 - protein, 12 aa, Saccharomyces cerevisiae
ISDAQNLFSFDL

SEQ ID NO: 4 - DNA, 32 bp, Artificial
  an artificially synthesized primer sequence
tacacatgtcatttcaccaacagttctttacc

SEQ ID NO: 5 - DNA, 42 bp, Artificial
  an artificially synthesized primer sequence
atctctagattatactttttgagcagcgtagttgtatttacc